(19) United States
(12) Patent Application Publication
(10) Pub. No.: US 2023/0052709 A1
(43) Pub. Date: Feb. 16, 2023

(54) TOPICAL ANTIVIRAL LIQUID FOR TREATMENT OF WARTS

(71) Applicants: Alexander Sepper, New York, NY (US); Dennis Tubian, New York, NY (US); John Navi, New York, NY (US)

(72) Inventors: Alexander Sepper, New York, NY (US); Dennis Tubian, New York, NY (US); John Navi, New York, NY (US)

(21) Appl. No.: 17/872,816

(22) Filed: Jul. 25, 2022

Related U.S. Application Data
(60) Provisional application No. 63/227,151, filed on Jul. 29, 2021.
(60) Provisional application No. 63/227,144, filed on Jul. 29, 2021.
(60) Provisional application No. 63/227,147, filed on Jul. 29, 2021.

(51) Int. Cl. A61K 33/34; A61K 9/06; A61K 9/00; A61P 17/12; A61K 31/194; A61K 31/191

(52) U.S. Cl. CPC A61K 33/34; A61K 9/06; A61K 9/0014; A61P 17/12; A61K 31/194; A61K 31/191

(57) ABSTRACT

A topical antiviral liquid for treatment of viral warts, which is a multi-component formula that contains a number of acids and salts in an aqueous solution, which liquid has been shown to have superior effectiveness in completely penetrating and removing wart tissue, while encouraging rapid regeneration of healthy tissue.

Relationship to other applications: The present non-provisional application claims priority to and the benefit of U.S. applications 63/227,151 filed 29 Jul. 2021, 63/227,144 filed 29 Jul. 2021 and 63/227,147 filed 29 Jul. 2021, all incorporated by reference herein.

### FIELD OF THE INVENTION

The field of the present invention relates to a topical liquid for treatment of viral warts.

### BACKGROUND OF THE INVENTION

Numerous innovations for wart treatments have been provided in the prior art (see table of FIG. 1), which will be described, infra, and which are incorporated herein in their entirety by reference thereto. Even though these innovations may be suitable for the specific individual purposes to which they address, however, they differ from the embodiments of the present invention. To date, there is no drug that can destroy or remove the causative factor responsible for skin viral warts (papillomavirus) from the body. Therefore, the treatment of human papilloma virus is limited to the local removal of warts or immunocorrective course of therapy. Despite the established viral etiology of skin warts, no treatment involving topical application of antiviral compounds has been found reliable and effective. There is a need for therapies and antiviral products can make a significant positive effect in the treatment of viral warts. The present invention addresses this need.

### BRIEF DESCRIPTION OF THE INVENTION

The present invention encompasses a topical antiviral liquid for treatment of viral warts, which is a multi-component formula that contains a number of acids and salts in an aqueous solution, which liquid has been shown to have superior effectiveness in completely penetrating and removing wart tissue, while encouraging rapid regeneration of healthy tissue. The present invention comprises a composition to treat warts, a method for making such a composition, and a method for using such a composition.

The invention is not limited to treatment of viral warts and may be directed to any form of external hyperplasic tissue.

Specifically, the composition comprises: copper (ii) nitrate trihydrate, cadmium nitrate tetrahydrate, oxalic acid dihydrate (C₂H₂O₄.2H₂O), lactic acid, pyruvic acid, acetic acid, nitric acid and distilled water.

The present invention comprises a method for making the above composition. The method for making the composition comprises standard steps of mixing various ingredients in a specified order.

The present invention comprises a method of therapeutic use is by topical application using a specific regimen.

The novelty of the composition rests in the combination of active components in specific amounts. This provides a composition with unexpectedly superior clinical results when compared to other known compositions and therapies.

### BRIEF DESCRIPTION OF THE DRAWINGS

FIG. 1 Prior-art methods of wart removal.

FIG. 2A-2H Treatment of wart on “frown area” of forehead.

FIG. 3 Treatment of two warts on forehead.

FIG. 4 Treatment of two warts on chin.

FIG. 5 Treatment of wart on finger.

FIG. 6 Treatment of two warts on hand.

FIG. 7 Treatment of plantar wart oh heel.

FIG. 8 Treatment of two warts on side of head.

FIG. 9A-9D Shows a flow chart of the steps of the method of making the composition.

FIG. 10 Shows a schematic of the treatment of step 1.

FIG. 11 Shows a schematic of the treatment of step 2.

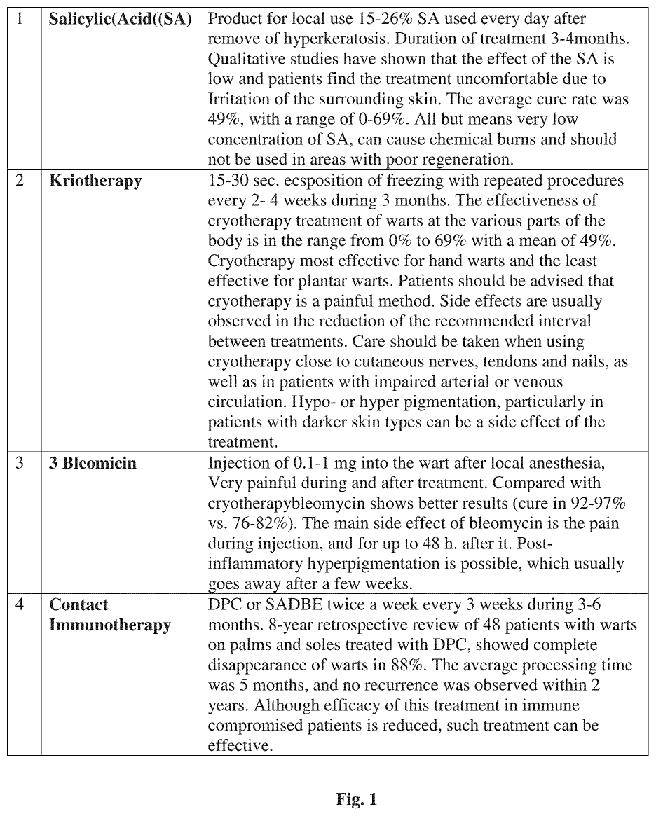

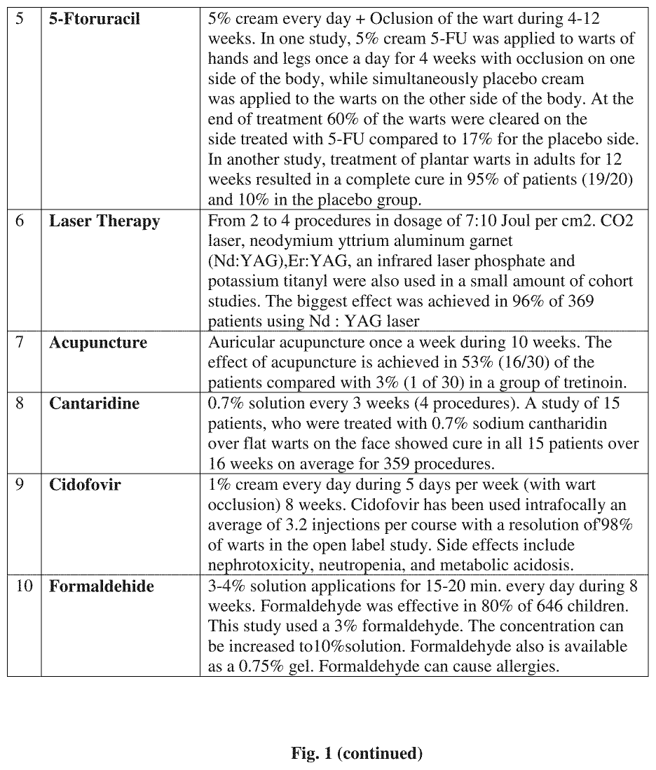

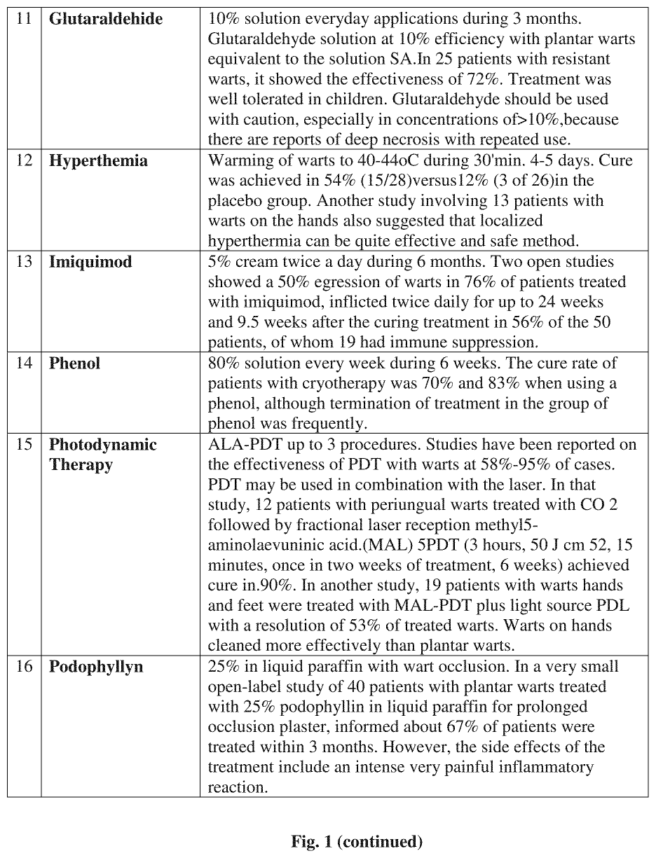

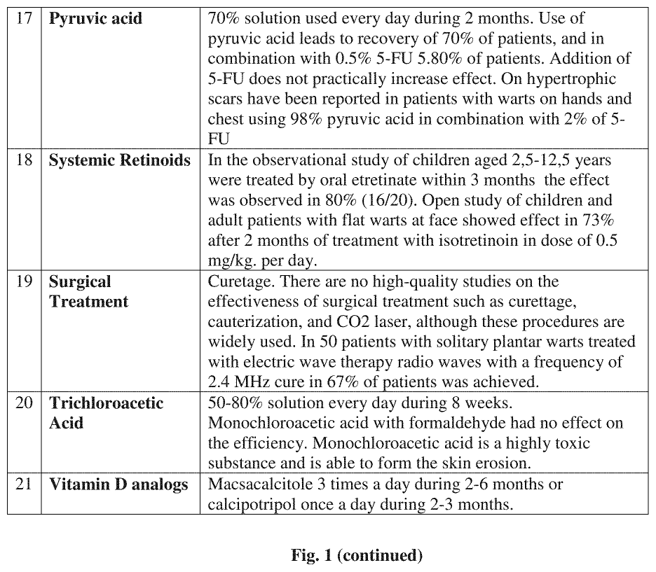

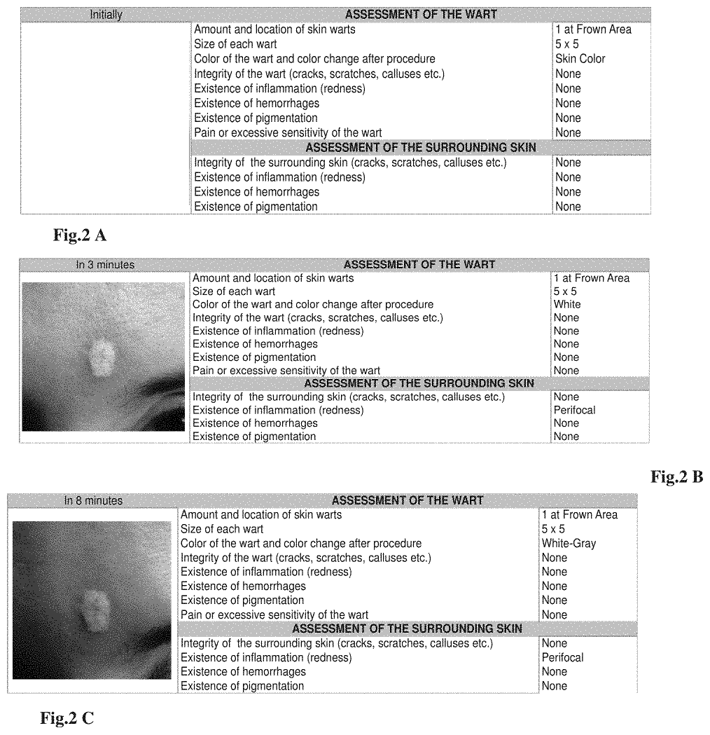

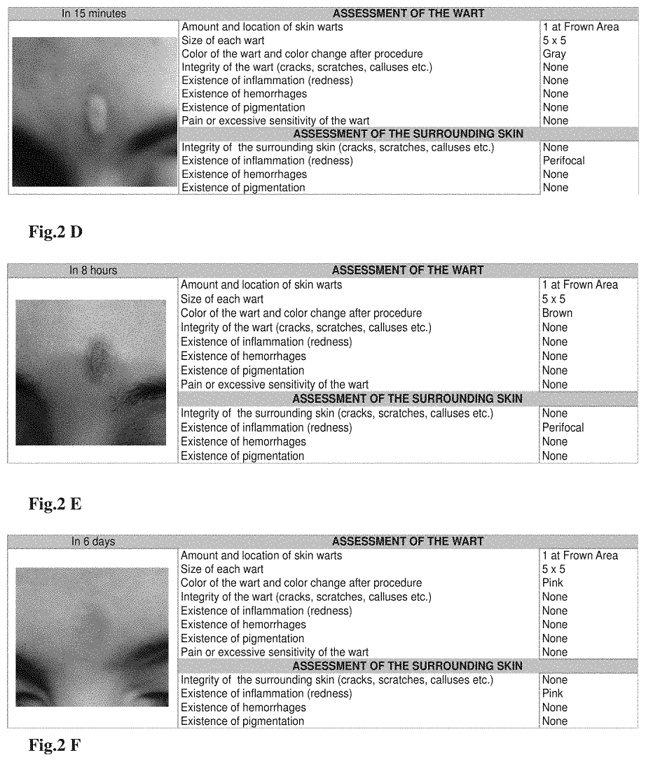

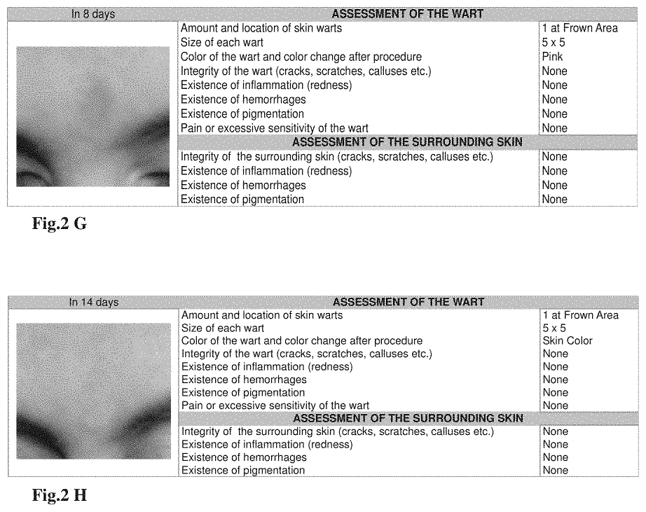

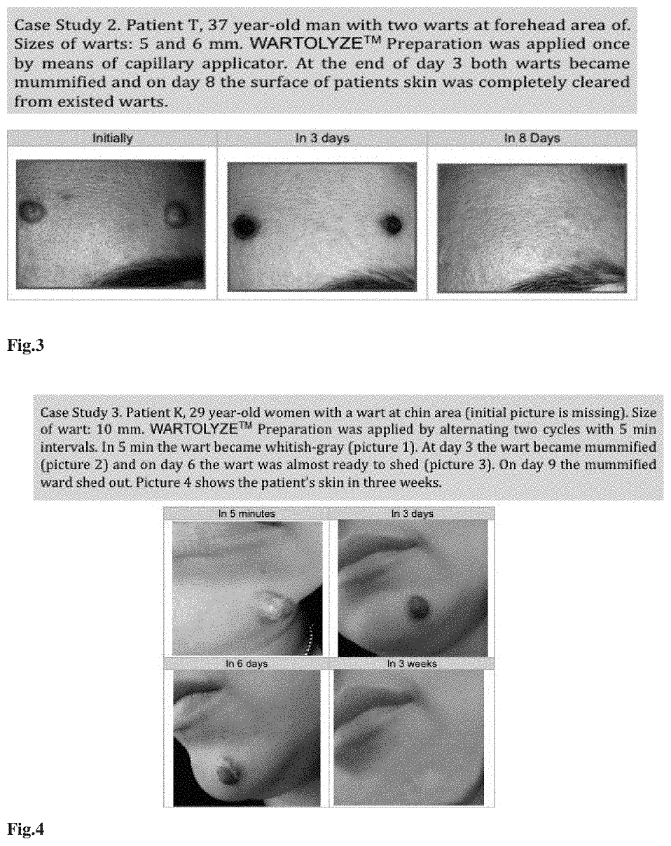

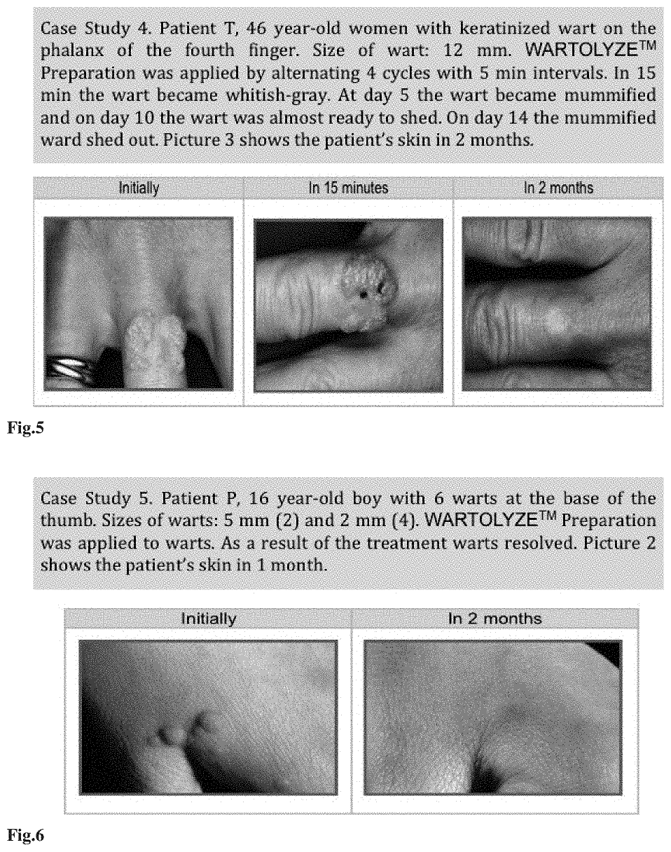

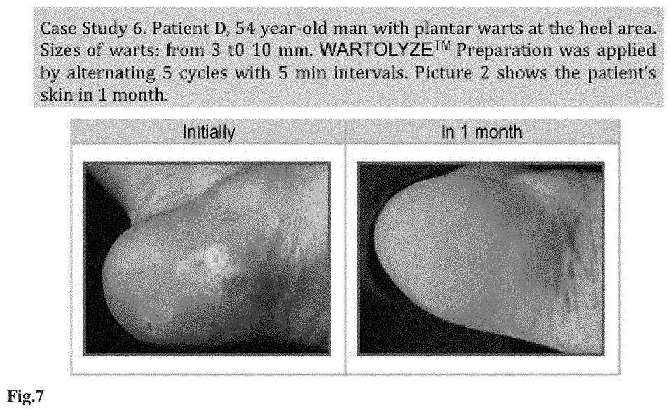

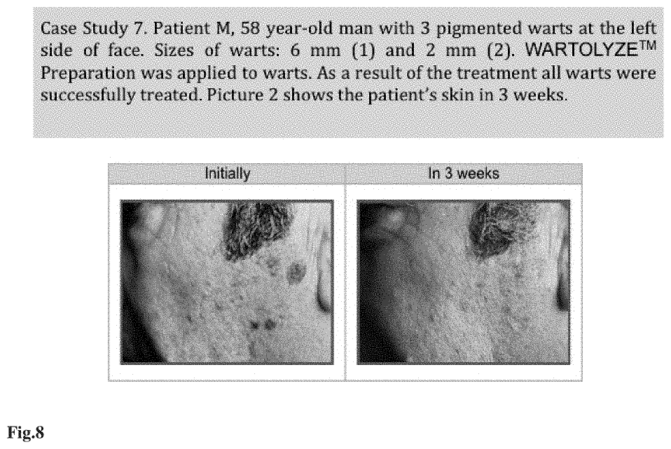

### DETAILED DESCRIPTION OF THE INVENTION

An object of the embodiments of the present invention is to provide a therapeutic formulation which is a topical antiviral liquid for the treatment of viral warts, which avoids the disadvantages of the prior art and, when tested, has been shown to provide unexpectedly superior clinical results when compared to other known compositions and therapies. Quantities discussed are generally given in wt/vol %, with all quantities +/−10%.

Therapeutic Composition

The therapeutic formulation (also referred to as the compound) of the invention is a multi-component formulation that contains a complex of the following carboxylic acids and certain salts of microelements incorporated into the primary liquid delivery system. In some embodiments salts may be halogenated, and in some embodiments polymers of carboxylic acids may be present.

The composition comprises the following therapeutic formulation in three parts, prepared and mixed as described herein (compound name followed by concentration range given in wt/vol %):

PART 1

| | |
|---|---|
| Copper (II)-nitrate trihydrate | 0.001-0.2% |
| Cadmium nitrate tetrahydrate | 0.23-1.3% |
| Oxalic acid dihydrate | 3.2-6.78% |
| Lactic acid 90% | 0.6-3.87% |
| Pyruvic acid | 0.022-1.94% |
| Acetic acid | 1.05-7.22% |
| Distilled water | 8.6-44.3% |

PART 2

| | |
|---|---|
| Nitric acid 65% | 14.6-72.45% |

PART 3

| | |
|---|---|
| Distilled water | 10.45-21.76% |

PART 4

| | |
|---|---|
| Distilled water | 1.5-31.4% |
| | (or to 100%) |

In a preferred embodiment the composition comprises the following therapeutic formulation in three parts, prepared and mixed as described herein (compound name followed by exemplary concentration used in tested formulation, given in wt/vol %, with all quantities +/−10%):

PART 1

| | |
|---|---|
| Copper (II)-nitrate trihydrate | 0.004 |
| Cadmium nitrate tetrahydrate | 0.432 |
| Oxalic acid dihydrate | 5.925 |
| Lactic acid 90% | 1.325 |
| Pyruvic acid | 0.042 |
| Acetic acid | 4.005 |
| Distilled water | 25.000 |

PART 2

| | |
|---|---|
| Nitric acid 65% | 50.7 |

PART 3

| | |
|---|---|
| Distilled Water | 12.567 (or |
| | to 100%) |
| TOTAL % | 100.000 |

Methods for Preparation of the Therapeutic Composition

Exemplary methods are detailed below. The invention is not limited to any of the specific methods. Methods may vary in terms of conditions, amounts, time or type of mixing to provide an equivalent therapeutic composition.

Method No. 1

The above therapeutic formulations of the invention are prepared as follows:

Part 1 Preparation

1) At room temperature, add all ingredients of Part 1 into a glass beaker.
2) Mix using a magnetic stirrer for 1 hour until the solution is clear.
3) Start homogenizing and micronizing the composition, for example using an UltraTurax™ homogenizer, or similar for 30-40 minutes.

Part 2 Preparation

1) To the homogenized solution of Part 1 add Part 2 (Nitric acid).
2) Mix using magnetic stirrer for 1 hour until the solution is clear.

Part 3 Preparation

1) Dilute Part 1 and Part 2 mixture with Distilled water.
2) Mix using magnetic stirrer for 1 hour until the solution is clear.

Method No. 2

In another embodiment therapeutic formulations of the invention are prepared as follows:

STEP 1: At room temperature, add one-by-one and mix together all components from PART 1. During stirring, the crystals gradually dissolve. Mix until complete dissolution of crystals;
STEP 2: In 30 minutes, add component from PART 2. The mixture warms to about 45° C. Mix the composition until it cools down to room temperature;
STEP 3: Add a component from PART 3. Stir the mixture in accordance to the algorithm: Final Amount of Product X 0.5=Mixing Time; and
STEP 4: Dilute with component from PART 4, and mix for additional 30 minutes.

Method No. 3

In another embodiment therapeutic formulations of the invention are prepared as follows:

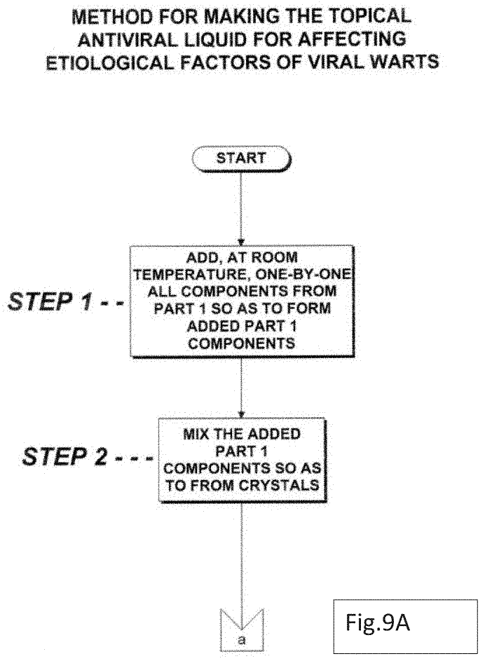

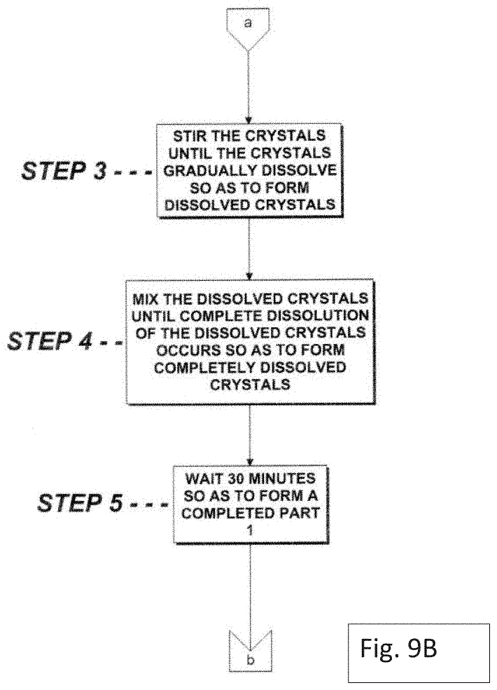

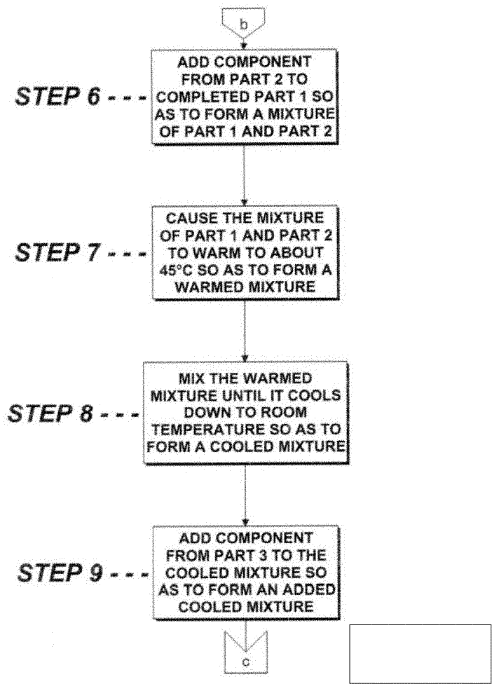

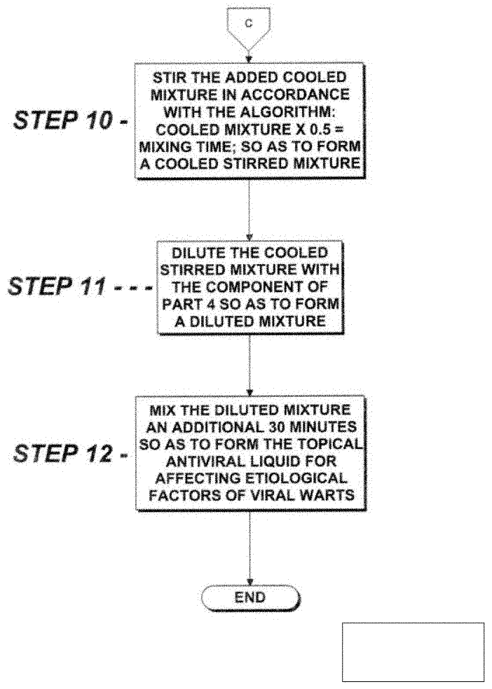

STEP 1: As shown in FIG. 9-A, add, at room temperature, one-by-one all components from PART 1 so as to form added PART 1 components;
STEP 2: As shown in FIG. 9-A, mix the added Part 1 components so as to from crystals;
STEP 3: As shown in FIG. 9-B, stir the crystals until the crystals gradually dissolve so as to form dissolved crystals;
STEP 4: As shown in FIG. 9-B, mix the dissolved crystals until complete dissolution of the dissolved crystals occurs so as to form completely dissolved crystals;
STEP 5: As shown in FIG. 9-B, wait 30 minutes so as to form a completed PART 1;
STEP 6: As shown in FIG. 9-C, add component from PART 2 to completed PART 1 so as to form a mixture of Part 1 and PART 2;
STEP 7: As shown in FIG. 9-C, cause the mixture of PART 1 and PART 2 to warm to about 45° C. so as to form a warmed mixture;
STEP 8: As shown in FIG. 9-C, mix the warmed mixture until it cools down to room temperature so as to form a cooled mixture;
STEP 9: As shown in FIG. 9-C, add component from PART 3 to the cooled mixture so as to form an added cooled mixture;
STEP 10: As shown in FIG. 9-D, stir the added cooled mixture in accordance with the algorithm: cooled mixture (in grams)×0.5=mixing time (in minutes), so as to form a cooled stirred mixture;
STEP 11: As shown in FIG. 9-D, dilute the cooled stirred mixture with the component of PART 4 so as to form a diluted mixture; and
STEP 12: As shown in FIG. 9-D, mix the diluted mixture for an additional 30 minutes so as to form the topical antiviral liquid for treatment of viral warts.

Methods for Using the Therapeutic Composition

The method of using a topical antiviral liquid for treatment of viral warts comprises the steps of:

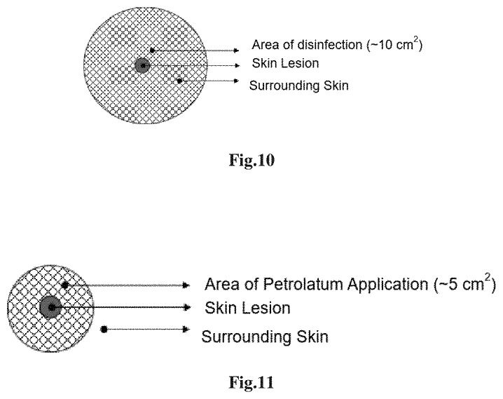

STEP 1: Clean, degrease, and disinfect the lesion and the surrounding skin with alcohol or ether, and then dry the area completely with a cotton swab or a gauze. See FIG. 10;
STEP 2: Apply with a Q-tip a thin layer of petrolatum ointment to the skin around the lesion to protect the skin from possible destructive effects of the treatment composition. See FIG. 11;
STEP 3: Use a glass capillary tube for accumulation of the therapeutic fluid in the right quantity, and accurate application of fluid on the surface of the wart or other skin formation undergoing the treatment;
STEP 4: Expect a mild transient erythema of the surrounding skin and development of a blanched (vasoconstricted) ring in 5-10 minutes, which requires no particular treatment;
STEP 5: Observe a desiccated dark brown scab that appears mummified;
STEP 6: Rejection of the scab on the spot of intervention; and
STEP 7: Observe usually no scar tissue, and in case of infection apply an antibiotic ointment.

Mechanism of action of the topical antiviral liquid for treating viral warts used by the method of using of the embodiments of the present invention is based largely on oxidation and a number of related chemical reactions of carboxylic acids and their intermediate reduction products used in balanced ratios in relatively low concentrations. The present invention shows more rapid and effective destruction of a skin lesion to which it is applied in the form of in-vivo fixation with the basic architecture of the lesion preserved rather than dissolution of the hydrolysis of protein peptide bonds. This contributes to the lesser damage of surrounding normal skin tissue because its destructive potential is more effectively neutralized or its penetration into surrounding normal tissue is more effectively blocked.

For the dosage and duration of application of the composition, the following clinical factors should be considered:
Place (localization) of the lesion;
Size of the cutaneous lesion;
Thickness of the cutaneous lesion;
Degree of lesion's callosity;
Usually, 0.05-0.1 ml of the solution is needed for the treatment;
Skin lesions of more than 10 mm diameter should only be treated after it has been ensured that only superficial skin lesions are concerned;
The number of lesions treated simultaneously and their total area should not exceed 4 to 5 and 5 cm2 respectively;
More than 0.25 ml should not be used during one sitting;
If mummification is unsatisfactory, a second treatment can take place 3 to 4 days later;
If a larger number of cutaneous lesions are involved, the treatment should be carried out in several sittings at 4-week intervals;
Treat only for a few minutes at first, to wait a short time for the reaction and then to proceed the treatment depending on the intensity of the reaction until the local discoloration is completely developed and the lesion begins to shrink slightly;
Local application of the composition leads to an immediate intravital fixation of the tissue with which it comes into contact. The extent of its effect is strictly limited to the treatment spot. The immediate effect is displayed in a white-yellowish to gray coloration of the treated spot. The tissue devitalized in this way dries up and changes color to dark brown upon increasing mummification;
The mummified scab detaches itself spontaneously after 2 to 5 weeks;
The healing generally takes place without complications, especially without secondary infection, quickly and without leaving ugly scars or distortion of the surrounding tissue;
The day after treatment, the patient can, e.g., wash, bath, or shower again;
The composition is not desorbed and has systemic effects; and
Sensitizing or allergic reactions have not been observed.

Clinical Comments and Notes on the Method

Quite often so-called “hanging” formations or pedinculated formations hanging on the intact skin surface is encountered. The application of caustic liquid on them is associated with frequent contact of the “wet” end of the hanging formation with healthy skin and, as a result of this contact, with an undesirable “burn” of surrounding healthy tissue. To prevent this, a 10% solution of aluminum-potassium alum (AlK(SO4)2) is applied to the hanging wart causing a hardening of the tissue and a kind of “erection” of the wart. As a result, the wart ceases to hang down and an accurate exposure of the therapeutic fluid and the prevention of a possible burn are provided.

One of the important questions of the method of treatment is the question of the precise and metered application of the caustic solution to the surface of the wart so as not to touch the surrounding healthy tissue and protect it from the caustic effect. This is an important methodological element that ensures the patient's comfortable condition during, and after, the procedure. To this end, a glass capillary tube for accumulation of therapeutic fluid in the right quantity and accurate application of fluid on the surface of the wart or other skin formation undergoing a medical procedure is used. Two types of glass tube diameter—0.5 and 0.8 mm provide a choice depending on the size of the skin formation. As one end of the capillary plunges into the fluid, automatic set takes place and a further dosed release of the required amount of fluid onto the surface of the skin formation;

In 10-20 seconds after the first application the wart begins to acquire a whitish-grayish color and becomes dense. After 2-3 minutes, the delivered solution penetrates the lesion and a color change develops reflecting intra-vital fixation of the lesion. The lesion darkens, step-by-step, from gray to dark gray;

In some cases (especially at small sizes of warts) one cycle is sufficient to initiate the devitalization of the wart. The number of applications, the time, and intensity of the discoloration depend on the type, size, and pigmentation of the lesion.

No more than 5-6 lesions should be treated at one time and their total area should not be more than 4.0 square sm.

The treatment is essentially painless, except for mild burning sensation, which can last 10-15 minutes;

A mild transient erythema of the surrounding skin and development of a blanched (vasoconstricted) ring is expected in 5-10 minutes, which requires no particular treatment;

In a few days, the lesion becomes a desiccated dark brown scab and appears mummified. If mummification is judged to be unsatisfactory in several days, the procedure may be repeated;

In general, this period lasts from 5 to 7 days. After the rejection of the scab on the spot of intervention remains an unobtrusive pinkish spot that in the next 20-30 days is covered by normal epidermis; and

In compliance with proper procedure, scar tissue usually is not appearing. In case of infection, application of antibiotic ointment is desirable.

Mechanisms of Action of the Therapeutic Composition

The mechanism of action of the topical antiviral liquid for treating viral warts of the embodiments of the present invention is based largely on oxidation and a number of related chemical reactions of carboxylic acids and their intermediate reduction products used in balanced ratios in relatively low concentrations.

The topical antiviral liquid for treating viral warts of the embodiments of the present invention shows rapid and effective destruction of a skin lesion to which it is applied in the form of in-vivo fixation with the basic architecture of the lesion preserved rather than destruction and hydrolysis of protein peptide bonds. The goal is to destroy the skin lesion (the morphological form of the lesion does not matter) in-vivo.

The compound provides effective destruction of the wart tissue and effective restoration of healthy tissue and rejuvenation of the skin. The goal is also to preserve the surrounding normal tissue and prevent its damage.

This contributes to the lesser damage of surrounding normal skin tissue because its destructive potential is more effectively neutralized or its penetration into surrounding normal tissue is more effectively blocked.

Objects and Advantage of the Invention

The topical antiviral liquid for treatment of viral warts provides the following features:

A virucidal preparation specificity having the ability to easily penetrate inside of the wart.

A preparation that does not irritate the skin and does not cause undesirable maceration, inflammatory, or coarse keratolytic process, which can lead to the spread of the virus to other skin areas.

A product that does not require intervention by medical staff, and can be used directly by the patient.

A product that is affordable and utilizes natural active ingredients.

The topical antiviral liquid for treatment of viral warts is a multi-component formula that, in some embodiments, contains a complex of the halogenated and polymerized carboxylic acids and certain salts of microelements incorporated into a liquid delivery system.

Further advantages of the present invention include:
Clinically proved 100% effective;
Suitable for all morphological and clinical types of viral warts;
Application in both the dermatological clinic and the beauty parlor;
Absence of pain during, and after, the procedure;
Absence of side effects and clinical complications;
The absence of virtual recurrences;
The absence of post treatment cosmetic defects such as scars, keloids, hyper- or hypopigmentation and other skin blemishes;
Aesthetic and cosmetic perfection;
High customer (patient) satisfaction;
Simplicity of implementation of the treatment;
The relative inexpensiveness, high revenue and availability of treatment;
No need to use expensive equipment;
Tangible commercial viability as compared to deposits and daily expenses;
No need for expensive facility set up;
Short duration of the procedure and the high rotation of patients (in one business day 40 and more patients can be treated);
The stability and reproducibility of the preparations used;
The mobility of the service and the possibility of creating a network of offices employing this technique; and
The patient feels no discomfort and in 1-2 hours after the procedure can follow the working or common regimes (take a shower, use makeup, continue the usual diet, etc.).

Clinical Results of Using the Topical Antiviral Liquid of the Invention

The therapeutic formulation of the invention has been clinically tested with considerable success. Results and case studies are shown in the Figures. The invention provides a composition with unexpectedly superior clinical results when compared to other known compositions and therapies.

General Disclosures

This specification incorporates by reference all documents referred to herein and all documents filed concurrently with this specification or filed previously in connection with this application, including but not limited to such documents which are open to public inspection with this specification. All numerical quantities mentioned herein include quantities that may be plus or minus 20% of the stated amount in every case, including where percentages are mentioned. As used in this specification, the singular forms “a, an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. For example, a composition “comprising” (or “which comprises”) ingredients A, B and C can contain only ingredients A, B and C, or can contain not only ingredients A, B and C but also one or more other ingredients. The term “consisting essentially of” and grammatical equivalents thereof is used herein to mean that, in addition to the features specifically identified, other features may be present which do not materially alter the claimed invention. The term “at least” followed by a number is used herein to denote the start of a range beginning with that number (which may be a range having an upper limit or no upper limit, depending on the variable being defined). For example, “at least 1” means 1 or more than 1, and “at least 80%” means 80% or more than 80%. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). When, in this specification, a range is given as “(a first number) to (a second number)” or “(a first number)-(a second number)”, this means a range whose lower limit is the first number and whose upper limit is the second number. For example, “from 40 to 70 microns” or “40-70 microns “means a range whose lower limit is 40 microns, and whose upper limit is 70 microns. The term ‘compounding’ in this disclosure is related to mixing one or more ingredients to provide a pharmaceutical preparation, i.e. a preparation designed to elicit a physiological effect.

Halogenated compounds are hydrocarbon compounds in which at least one hydrogen atom is replaced by a halogen atom, such as chlorine. When we talk about polymerization, we mean that it is a form of polymerization where smaller molecules or monomers react with each other to form larger structural units (usually polymers).

When discussing the amount of a compound in a final LIQUID formulation, we may use the relative units of Percent Volume (v/v). Typically, percent volume written as ‘% Vol’ or ‘Volume percent’ or ‘volume/volume percent’ or ‘v/v %’. This term is used when preparing solutions of liquids. Volume percent is defined as: v/v %=[(volume of solute)/(volume of solution)] x 100%. Note that volume percent is relative to the volume of solution, not the volume of solvent. For example, wine is about 12% v/v ethanol. This means there is 12 ml ethanol for every 100 ml of wine. It is important to realize liquid and gas volumes are not necessarily additive. If you mix 12 ml of ethanol and 100 ml of wine, you will get less than 112 ml of solution.

Alternatively, in a liquid formulation, quantities can be given in terms of relative percent concentration by weight of solute in a solution by volume (sometimes given as the relative units wt/vol % or % wt/vol or wt/vol or wt/v or w/v). To determine the weight percent of a solution, divide the mass of solute by mass of the solution (solute and solvent together) and multiply by 100 to obtain percent. The percent by weight formula=gram of solute/100 g of solution.

Quantities in a solid formulation are given wt/wt %, also called weight fraction, sometimes shown as wt/wt or % wt/wt or wt/wt %.

Alternatively and as frequently used in this disclosure, any mixtures such as solids in liquids, liquid in liquid and solids mixed with solids can also be expressed as % wt/wt. The weight of water is generally about 1 g/ml, so wt/vol and wt/wt is often similar or the same for a generally aqueous solution.

1. A composition for the treatment of warts comprising a plurality of components mixed together in an aqueous solution such that the resulting composition is a clear liquid, the components comprising:
copper nitrate trihydrate,
cadmium nitrate tetrahydrate,
oxalic acid dihydrate,
lactic acid,
pyruvic acid,
acetic acid,
nitric acid, and
distilled water.

2. The composition of claim 1 wherein the components consist only of the following:
copper nitrate trihydrate,
cadmium nitrate tetrahydrate,
oxalic acid dihydrate,
lactic acid,
pyruvic acid,
acetic acid,
nitric acid, and
distilled water.

3. The composition of claim 1 wherein the components are present in the following concentration ranges (wt/vol %):
Copper nitrate trihydrate: 0.001-0.20
Cadmium nitrate tetrahydrate: 0.23-1.30
Oxalic acid dihydrate: 3.20-6.78
Lactic acid (90% solution): 0.60-3.87
Pyruvic acid: 0.022-1.94
Acetic acid: 1.05-7.22
Nitric acid (65% solution): 14.6-62.80, and
wherein the remaining balance is provided by water.

4. The composition of claim 1 wherein the components are present in the following concentration ranges (wt/vol %):
Copper nitrate trihydrate: up to 1.00
Cadmium nitrate tetrahydrate: up to 2.00
Oxalic acid dihydrate: up to 10.00
Lactic acid (90% solution): up to 5.00
Pyruvic acid: up to 3.00
Acetic acid: up to 12.00
Nitric acid (65% solution): up to 75.00, and
wherein the remaining balance is provided by water.

5. The composition of claim 1 wherein the components are present in the following concentration ranges (wt/vol %):
Copper nitrate trihydrate: up to 0.20
Cadmium nitrate tetrahydrate: up to 1.30
Oxalic acid dihydrate: up to 6.78
Lactic acid (90% solution): up to 3.87
Pyruvic acid: up to 1.94
Acetic acid: up to 7.22
Nitric acid (65% solution): up to 50.00, and
wherein the remaining balance is provided by water.

6. A method for making a liquid composition for the treatment of warts, the method comprising the following steps:
(A) providing the following parts (in final wt/vol %, +/−10%):
PART 1

| | |
|---|---|
| copper nitrate trihydrate | 0.004 |
| cadmium nitrate tetrahydrate | 0.432 |
| oxalic acid dihydrate | 5.925 |
| lactic acid 90% | 1.325 |
| pyruvic acid | 0.042 |
| acetic acid | 4.005 |
| distilled water | 25.000 |

PART 2

| | |
|---|---|
| nitric acid 65% | 50.7 |

PART 3

| | |
|---|---|
| distilled Water | 12.567 |

(B) combining said parts as follows:
(1) at room temperature, adding all ingredients of Part 1 into a container and mixing until the solution is clear, then homogenize the solution for 30-40 minutes;
(2) adding Part 2 to the solution and mixing for about 1 hour until the solution is clear; and
(3) diluting with distilled water; and mixing for about 1 hour until the solution is clear;
thereby producing a liquid composition for the treatment of warts.

7. The method of claim 6 wherein the components of part 1 are added one-by-one, at room temperature, into a container.

8. The method of claim 6 wherein, in step (B)(2), the mixture is exothermic and warms to about 45° C. and is then cooled to room temperature before proceeding to step (B)(3).

9. The method of claim 6 wherein, in step (B)(2), the mixture is stirred for a time according to the formula: Final Amount of Product (g)×0.5=Mixing Time (min).

10. A method of using a topical antiviral liquid for treatment of viral warts comprising the steps of providing a subject with a lesion in need of treatment, and:
(1) clean, degrease, and disinfect the wart and the surrounding skin,
(2) apply a thin layer of hydrophobic ointment to the skin around the lesion to protect the skin from possible destructive effects of the treatment composition,
(3) use a glass capillary tube for accumulation of the antiviral liquid in a desired quantity and apply said antiviral liquid to the surface of the wart such that a transient erythema of the surrounding skin and development of a blanched, vasoconstricted ring is observed 5-10 minutes,
(4) observe the formation of desiccated dark brown scab over time, under which new healthy tissue will form.

11. The method of claim 10 wherein the number of warts treated simultaneously and their total area does not exceed 5 and 5 cm2 respectively.

12. The method of claim 10 wherein 0.05-0.1 ml of said topical antiviral liquid is applied per treatment.

13. The method of claim 10 wherein no more than 0.25 ml of said topical antiviral liquid is applied per treatment.

14. The method of claim 10 wherein said treatment is repeated over a number of days.

15. The method of claim 14 wherein said treatment is repeated at 4-week intervals.

16. The method of claim 10 wherein said glass capillary tube has a diameter of between 0.5 mm and 0.8 mm.

17. The composition of claim 1 wherein the components are present in the following concentration ranges (wt/vol %):

| | |
|---|---|
| Copper (II)-nitrate trihydrate | 0.004 |
| Cadmium nitrate tetrahydrate | 0.432 |
| Oxalic acid dihydrate | 5.925 |
| Lactic acid 90% | 1.325 |
| Pyruvic acid | 0.042 |
| Acetic acid | 4.005 |
| Nitric acid 65% | 50.7 |
| Distilled Water | to 100% |
